(19) United States
(12) Patent Application Publication
(10) Pub. No.: US 2007/0168000 A1
(43) Pub. Date: Jul. 19, 2007

(54) Apparatus and method for cooling lasers using insulator fluid

(76) Inventors: Gemunu S. Happawana, Plano, TX (US); Arye Rosen, Cherry Hill, NJ (US); Walter M. Janton, Bellmawr, NJ (US); Gary A. Evans, Plano, TX (US)

Correspondence Address: BAKER BOTTS L.L.P., 2001 ROSS AVENUE, SUITE 600, DALLAS, TX, 75201-2980, US

(21) Appl. No.: 11/331,947

(22) Filed: Jan. 13, 2006

(51) Int. Cl. A61N 5/06

(52) U.S. Cl. 607/88

(57) ABSTRACT

A semiconductor device system includes a chamber, one or more semiconductor devices disposed within the chamber and operable to emit light, and an insulator fluid disposed within the chamber. The insulator fluid may be in contact with the semiconductor devices and operable to decrease the temperature of the semiconductor devices. The insulator fluid may comprise deionized water.

### TECHNICAL FIELD

This invention relates generally to the field of optics and, more specifically, to an apparatus and a method for cooling semiconductor devices with insulator fluid.

### BACKGROUND

Many types of cancer can be treated by photodynamic therapy (PDT), which destroys cancer cells through the use of light in combination with a photosensitive drug. The drug is administered to a patient many hours before treatment to allow the drug to travel through the blood stream. The drug accumulates more in cancer cells than in healthy tissue, and is activated by light. Illuminating the cancerous area causes the drug to react with oxygen and kills the cancer cells, with little damage to surrounding healthy tissue. As a result, the cumulative toxicity associated with repeated ionizing radiation treatments can be largely avoided with PDT.

### SUMMARY OF THE DISCLOSURE

In accordance with the present invention, disadvantages and problems associated with previous techniques for cooling semiconductor devices may be reduced or eliminated.

In accordance with an embodiment, a semiconductor device system includes a chamber, one or more semiconductor devices disposed within the chamber and operable to emit light, and an insulator fluid disposed within the chamber. The insulator fluid may be in contact with the one or more semiconductor devices and operable to decrease the temperature of the one or more semiconductor devices.

In another embodiment, the insulator fluid may comprise deionized water. In another embodiment, the semiconductor device system may also include a cover insertable into a passage. The chamber may be disposed within the cover. In another embodiment, the semiconductor device system may also include a substrate disposed within the chamber and one or more submounts coupled to a surface of the substrate. In this embodiment, a semiconductor device of the one or more semiconductor devices may be coupled to the one or more submounts.

In accordance with an embodiment, the chamber of the semiconductor device system may also include a flexible conduit wherein insulator fluid is disposed. In another embodiment, the chamber of the semiconductor device system may also include a second flexible conduit disposed within the first flexible conduit wherein the insulator fluid enters the chamber through a flexible conduit of the first and second flexible conduits and leaves the chamber through the other flexible conduit of the first and second flexible conduits.

In one embodiment, the semiconductor device system may also include one or more focusing elements. Each focusing element may include a surface operable to change shape in response to a pressure change in the insulator fluid. The surface may also focus the light emitted from a semiconductor devices of the one or more semiconductor devices.

In one embodiment, the semiconductor device system may also include a heating element disposed within the chamber. The heating element may be operable to increase the temperature of an area disposed outwardly from the chamber.

In one embodiment, the semiconductor device system may also include an oxygen detector disposed within the chamber. The oxygen detector may be operable to detect oxygen in an area disposed outwardly from the chamber.

In one embodiment, a semiconductor device of the one or more semiconductor devices of the semiconductor device system may include a light scattering element. The light scattering element may be operable to scatter light emitted from the semiconductor device.

Certain embodiments of the invention may provide one or more technical advantages. A technical advantage of one embodiment may be that insulator fluid may be in direct contact with a semiconductor laser to cool the laser. The direct contact may allow the fluid to cool many sides of the laser using convection and/or conduction cooling. Another technical advantage of one embodiment may be that the cooling fluid need not be enclosed in separate inflexible tubing. The elimination of the tubing may allow for a more flexible and maneuverable device. Another technical advantage of one embodiment may be that cooling the laser may improve laser efficiency, which may allow for higher power operation.

Certain embodiments of the invention may include none, some, or all of the above technical advantages. One or more other technical advantages may be readily apparent to one skilled in the art from the figures, descriptions, and claims included herein.

### BRIEF DESCRIPTION OF THE DRAWINGS

For a more complete understanding of the present invention and its features and advantages, reference is now made to the following description, taken in conjunction with the accompanying drawings, in which:

FIG. 1 is a schematic of a patient receiving photodynamic therapy with a balloon catheter having a semiconductor laser system cooled using insulator fluid according to an embodiment of the invention;

FIG. 2 is a schematic of the balloon catheter illustrating the semiconductor laser system disposed therein;

FIG. 3 is a partial side view of the semiconductor laser system of FIG. 2 illustrating cooling the lasers using insulator fluid according to an embodiment of the invention;

FIG. 4 is a cross-section of the semiconductor laser system of FIG. 3 illustrating one embodiment of the cooling system; and

FIG. 5 is a partial elevation view of a semiconductor laser having a pair of light scattering elements according to an embodiment of the invention.

### DETAILED DESCRIPTION OF THE DRAWINGS

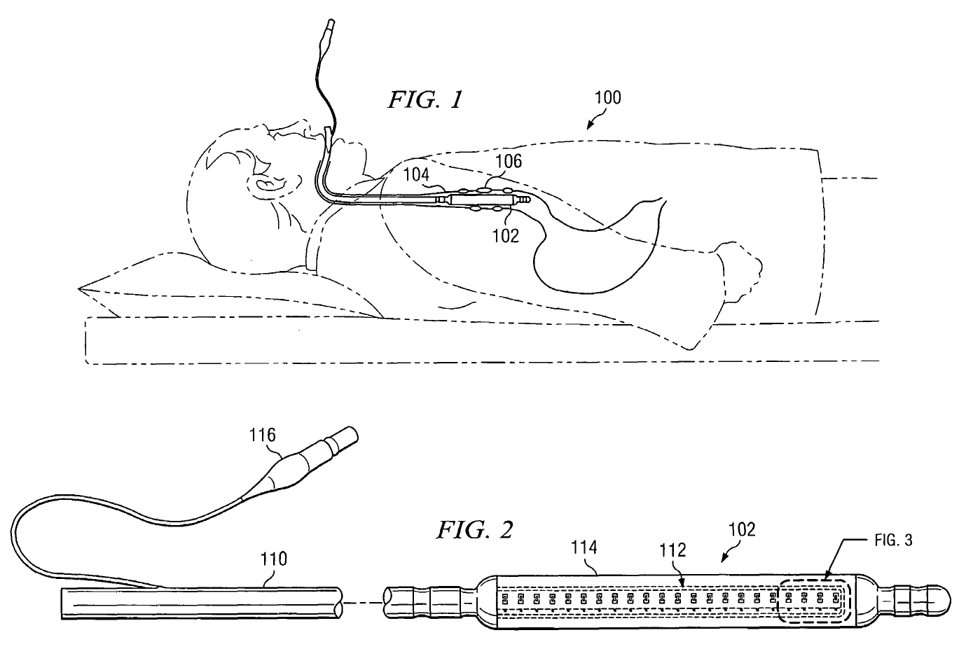

Embodiments of the present invention and its advantages are best understood by referring to FIGS. 1 through 5 of the drawings, like numerals being used for like and corresponding parts of the various drawings.

FIG. 1 is a schematic of a patient 100 receiving photodynamic therapy (PDT) with a balloon catheter 102 having a semiconductor laser system cooled using an insulator fluid according to one embodiment of the invention. In this embodiment, the insulator fluid may circulate in direct contact with semiconductor lasers to maintain and/or reduce the temperature of semiconductor lasers disposed within the semiconductor laser system.

Many types of diseases or conditions can be treated by photodynamic therapy (PDT), which destroys the affected cells through the use of light in combination with a photosensitive drug. In the illustrated example, balloon catheter 102 is inserted into an esophagus 104 of patient 100 in order to treat Barrett's esophagus, which is a pre-cancerous lesion 106 of the esophagus lining. Balloon catheter 102 may be inserted in any suitable passageway to treat diseases or conditions in other parts of patient 100. In this example, the photosensitive drug administered to patient 100 accumulates more in affected cells such as pre-cancerous lesion 106 than in healthy tissue.

Balloon catheter 102 includes a plurality of semiconductor lasers that emit light to activate the photosensitive drug in the pre-cancerous lesion 106 by converting O₂ in the cancerous tissue to a highly reactive state of the oxygen known as singlet oxygen. The drug may be activated with little damage to surrounding healthy tissue. As a result, the cumulative toxicity associated with repeated ionizing radiation treatments may be avoided with PDT. Although the semiconductor laser system is illustrated for use in treating esophagus 104, it will be recognized by those of ordinary skill in the art that the semiconductor laser system could be used for any suitable laser application.

FIG. 2 is a schematic of balloon catheter 102 illustrating a semiconductor laser system 112 disposed therein according to an embodiment of the invention. Balloon catheter 102, which may be any suitable balloon catheter, may include a cover coupled to an inflation line 116 via any suitable conduit 110. For semiconductor laser system 112, any suitable cover may be used. The cover may be of any suitable size and shape, and may be formed from any suitable material. The cover may be designed to be inserted into a passageway of patient 100.

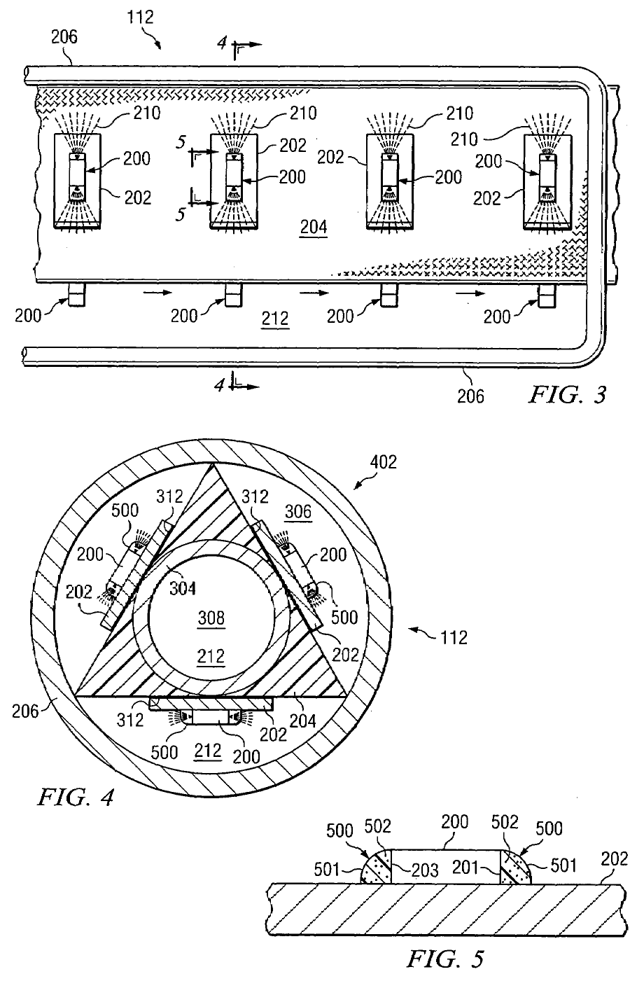

One example of the cover is an inflatable balloon 114. Inflatable balloon 114 may be any suitably sized balloon formed from any suitable material. Generally, after inflatable balloon 114 is inserted into esophagus 104 of patient 100 as shown in FIG. 1, inflation line 116 is utilized to inflate inflatable balloon 114 by delivering air or other suitable gas through conduit 110 into inflatable balloon 114. Laser system 112, which is described in greater detail below in conjunction with FIGS. 3-5, may then be utilized to deliver light to the inside of the esophagus 104 in order to activate a photosensitive drug in the blood of patient 100, which is given to the patient some period of time before the treatment. Although not illustrated in FIG. 2, laser system 112 includes one or more wires coupled thereto that extend through conduit 110 to the outside of the body of patient 100 in order to power the individual lasers.

Currently, there are two approaches to delivering light from lasers to cancer cells: by direct illumination from a laser and by indirect illumination from an optical fiber coupled to the laser. In the embodiments illustrated in FIGS. 1 and 2, patient 100 is receiving PDT by direct illumination from semiconductor lasers enclosed in balloon catheter 102. In other embodiments using indirect illumination, light from a semiconductor laser may be coupled to an optical fiber that includes a Bragg grating or diffusing region over a portion of the fiber to couple the light out and direct it towards the cancer cells. In some cases, the interior of the lung or brain may be treated with relative ease using indirect illumination method because the optical fiber may have a small diameter (100 to 500 μm) and may be easier to insert. Coupling high power light from many lasers into a single fiber, however, requires precision alignment techniques that may degrade efficiency in an indirect illumination system. The cooling method of certain embodiments may be used to cool the lasers of both direct and indirect illumination systems. For example, in an indirect illumination system, the cooling method may cool the lasers outside the body of the patient to increase the efficiency of the power introduced into the fibers.

FIG. 3 is a partial side view of semiconductor laser system 112 of FIG. 2 illustrating cooling semiconductor lasers 200 using insulator fluid according to an embodiment of the invention. In this embodiment, insulator fluid may circulate through the chamber substantially surrounding semiconductor lasers 200 to reduce the temperature of the lasers by conduction and/or convection cooling.

Semiconductor lasers 200 may be disposed within a chamber 212. Chamber 212 may refer to a volume that substantially surrounds semiconductor lasers 200. In the illustrated embodiment, the chamber includes a first flexible conduit 206, which is generally disposed around substrate 204 and semiconductor lasers 200. First flexible conduit 206 may be coupled to substrate 204 in any suitable manner. In one embodiment, an epoxy may be utilized.

In the illustrated embodiment, semiconductor laser system 112 includes a plurality of semiconductor lasers 200 each coupled to respective submounts 202. Submounts 202, in the illustrated embodiment, are rectangular shaped elements formed from any suitable material. Submounts 202, however, may have any suitable shape and may be formed from any suitable material having any suitable thickness. Semiconductor lasers 200 may be coupled to submounts 202 in any suitable manner. In some embodiments, semiconductor lasers 200 are soldered to submounts 202 with indium or gold/tin solder, or are epoxied to submounts 202.

After semiconductor lasers 200 are coupled to submounts 202, submounts 202 may be coupled to substrate 204. In one embodiment, substrate 204 may be formed from a copper braid; however, the present invention contemplates other materials and forms for substrate 204. Generally, substrate 204 may be any suitably shaped strip of material having any suitable thickness that is utilized as a base for submounts 202 having lasers 200 thereon.

Submounts 202 may be coupled to substrate 204 in any suitable manner. In one embodiment, an epoxy may be utilized. As illustrated in FIG. 2, substrate 204 generally runs almost the full length of inflatable balloon 114. Substrate 204, however, may have any suitable length. In addition, any suitable number of submounts 202 and corresponding semiconductor lasers 200 may be disposed on substrate 204 at any suitable spacing. The spacing of semiconductor lasers 200 may be determined in any suitable manner, for example, based on the proper illumination of light within esophagus 104 of patient 100.

Semiconductor lasers 200 may represent any suitable lasers, semiconductor or otherwise, that emit light of any suitable wavelength. In one embodiment, semiconductor lasers 200 are edge-emitting lasers that emit light with a wavelength of approximately 635 nanometers ±5 nanometers. This wavelength range may be utilized for the treatment of Barrett's esophagus with photosensitive agent PHOTOFRIN manufactured by WYETH-AYERST LEDERLE, INC. Other wavelengths may be used for other photosensitive agents.

Semiconductor lasers 200 may be of any suitable size and shape and may comprise any suitable semiconductor material. As illustrated in FIG. 3, semiconductor lasers 200 are generally rectangular (or square) in shape and are constructed in such a manner as to emit light 210 out of two facets 201 and 203. Lasers 200 may have any suitable farfield half power beam divergence, for example, 45 degrees of light divergence. Facets 201 and 203 have light scattering elements 500 coupled thereto in order to scatter light 210 to achieve increased farfield divergence of approximately 180 degrees and more uniform distribution of light within esophagus 104 of patient 100. Light scattering elements 500 are described in greater detail below in conjunction with FIG. 5.

Although not illustrated in FIG. 3, semiconductor lasers 200 are coupled to one another with one or more wires or flexible circuit traces. In order for laser system 112 to be flexible, the wires may be coupled within an epoxy or other suitable material between lasers 200. In addition, the wires may be arranged in any suitable manner. As an example, the wires may be arranged in a conventional series connection. As another example, the wires may be arranged such that different laser sections can be independently turned on or off.

Semiconductor lasers may convert electrical power to optical power with an efficiency as high as approximately 70%, but more typically at approximately 30% for semiconductor lasers with satisfactory beam and spectral quality. As a result, 30% to 70% of the electrical power is converted to heat. The heat may severely limit the output power and lifetime and reliability of the lasers and may also cause undesirable wavelength shift. Moreover, at shorter wavelengths (630 to 655 nm) and at longer wavelengths (1300 to 1700 nm), the output power and the efficiency may be lower. Consequently, even more heat may be generated, which in turn may lower the efficiency at high drive currents.

A cooling system may be used to reduce the temperature of semiconductor lasers 200 or maintain the temperature of semiconductor lasers 200 at any suitable temperature, for example, a temperature at which wavelength shift is avoided. According to one embodiment, a fluid may circulate through first flexible conduit 206 in direct contact with semiconductor lasers 200 in order to maintain or reduce the temperature of semiconductor lasers 200. By placing the fluid in direct contact with semiconductor lasers 200, the fluid maintains or decreases the temperature of semiconductor lasers 200 by convection, conduction, or both convection and conduction heat transfer.

The fluid may be applied to semiconductor lasers 200 in any suitable manner. In some examples, the fluid may be applied to semiconductor lasers 200 to cool the lasers from more than one side. In one example, semiconductor lasers 200 may be submerged in the fluid so that the lasers are cooled from many sides. The fluid may circulate through chamber in any suitable manner. In one embodiment, the fluid may circulate through first flexible conduit 206. In another embodiment, the fluid may flow in through first flexible conduit 206 and out through second flexible conduit 304. In yet another embodiment, the fluid may flow in through second flexible conduit 304 and out through first flexible conduit 206.

The fluid may comprise any suitable fluid. In some embodiments of the cooling system, the cooling fluid may include an insulator fluid. The insulator fluid may be any fluid that conducts little or no electricity, sound, and/or vibration. In some embodiments, the cooling system circulates an electrically-resistant insulator fluid in direct contact with semiconductor lasers 200 to avoid an electrical short in semiconductor laser system 112. In some cases, the insulator fluid may be deionized water for introduction into patient 100.

In one embodiment, the insulator fluid may include deionized water, for example, deionized water with 18 Mega Ohm resistance. Deionized water (DI) is water that has been substantially purified from ions by an ion exchange process. Deionized water has high electrical resistance properties and is considered non-toxic to the human body.

Certain embodiments of the invention may provide one or more technical advantages. A technical advantage of one embodiment may be that deionized water may be in direct contact with a semiconductor laser to cool the laser. The direct contact may allow the fluid to cool many sides of the laser using convection and/or conduction cooling. Another technical advantage of one embodiment may be that the cooling fluid need not be enclosed in separate inflexible tubing. The elimination of the tubing may allow for a more flexible and maneuverable device. Another technical advantage of one embodiment may be that cooling the laser may improve laser efficiency and wavelength stability, which may allow for higher power operation.

It will be recognized by those of ordinary skill in the art that direct cooling with an insulator fluid may improve the performance of any suitable semiconductor device such as a semiconductor laser, a light emitting diode, or a microwave device. It will also be recognized by those of ordinary skill in the art that direct cooling with deionized water may be used in any suitable laser application including PDT, welding, optical pumping, biological, and photochemical applications.

FIG. 4 is a cross-section of semiconductor laser system 112 illustrating one embodiment of the cooling system. In the illustrated embodiment, semiconductor laser system 112 includes chamber 212 comprised of first flexible conduit 206 and second flexible conduit 304, semiconductor lasers 200 for emitting light outside chamber 212, and a fluid circulating through chamber 212 for cooling semiconductor lasers 200.

In one embodiment, the cooling fluid may flow into semiconductor laser system 112 through one of first or second flexible conduits 206, 304 and out of laser system 112 through the other of first or second flexible conduits 206, 304. For example, the cooling fluid may flow in through first volume 306 in contact with semiconductor lasers 200 and may flow out through second volume 308 removing heat from semiconductor lasers 200. In this example, the cooler fluid entering semiconductor laser system 112 flows along semiconductor lasers 200.

Second flexible conduit 304 may be disposed within triangular substrate 204. First flexible conduit 206 and second flexible conduit 308 may be of any suitable size and shape and may be formed from any suitable material, such as a material that provides specified flexibility. All of these components may be coupled together with an epoxy 312 or other suitable coupling methods. First flexible conduit 206 forms a first volume 306. A first volume 308 may be formed by the inner surface of first flexible conduit 304. A second volume 308 may be formed between second flexible conduit 304 and a structure comprising semiconductor lasers 200, submounts 312, substrate 204, and first flexible conduit 304.

The cooling system of semiconductor laser system 112 may include a liquid or gas cooling fluid circulating through first volume 306 and/or second volume 308 to maintain or reduce the temperature of semiconductor lasers 200. In one embodiment of the cooling system, the fluid may circulate in first volume 306 in direct contact with semiconductor lasers 200.

Semiconductor laser system 112 also includes triangular substrate 204 and submounts 202 coupled to the faces of triangular substrate 204. Each submount 202 has a respective semiconductor laser 200 coupled thereto. Each semiconductor laser has a light scattering element 500 coupled thereto. Although a generally triangular configuration of laser system 112 is illustrated in FIG. 4, the present invention contemplates other suitable configurations for laser system 112.

In some embodiments, semiconductor laser system 112 may include a focusing element that adjusts the focal length of a semiconductor laser 200 to focus light. In one embodiment, the focusing element may be disposed within first volume 306 between a particular semiconductor laser 200 and the inner wall of first flexible conduit 206. The focusing element may be any apparatus operable to change the focal length. In one example, the focusing element includes a liquid lens having a diaphragm made of any material suitable for changing convexity of a surface of the diaphragm. In this embodiment, laser system 112 may change the pressure of the fluid to control the convexity of the diaphragm. By changing the focal length, the laser system 112 may focus light on particular areas and intensify the light on particular treatment areas of patient 100.

In one embodiment, semiconductor laser system 112 may include a heating element to heat the area being treated. In general, heating cancerous tissue brings blood circulation and oxygen to the tissue, which in turn makes the PDT treatment more effective. In some cases, a heating element may be used to reduce treatment time by an order of magnitude. The heating element may be located in any part of semiconductor laser system 112 that would be suitable for heating the area being treated. In one case, the heating element may be disposed within balloon catheter 102. The heating element may include any suitable device, circuitry, or chemicals to heat the area being treated. In one example, the heating element may include microwave circuitry.

In another embodiment, laser system 112 may include an oxygen detector operable to detect the amount of oxygen in the area being treated. An oxygen detector may be any suitable device for detecting oxygen, for example, an electrochemical oxygen detector. In PDT, the more oxygen content in the area being treated, the less light may be necessary to maintain the same level of treatment. In one embodiment, semiconductor laser system 112 may control light output from semiconductor lasers 200 based on the amount of oxygen detected in the area being treated. In one example, laser system 112 may reduce or increase the amount of light from a particular semiconductor laser 200 in response to the amount of oxygen detected in the area where the particular laser 200 is focused. In another example, certain semiconductor lasers 200 may be turned off/on according to the amount of oxygen detected in the areas corresponding to the certain semiconductor lasers 200.

The oxygen detector may have any suitable location. In one case, the oxygen detector may be disposed within balloon catheter 102.

FIG. 5 is a partial elevation view of a particular semiconductor laser 200 having a pair of light scattering elements 500 coupled thereto according to an embodiment of the invention. Light scattering elements 500 are each formed from a polymer 501, and particles 502 of suitable types are dispersed within polymer 501.

Polymer 501 may be any suitable light transparent polymer. Other suitable light transparent materials may be used in place of polymer 501. Polymer 501 may have any suitable shape and may be coupled to laser 200 in any suitable manner. In a particular embodiment, polymer 501 having particles 502 therein is deposited directly onto facets 201 and 203 of semiconductor laser 200 using inkjet deposition. Other deposition methods may be used. In one embodiment, facets 201 and 203 may have a facet coating formed of one or more passivation layers (not explicitly shown) formed outwardly therefrom before light scattering elements 500 are coupled thereto. Any suitable dielectrics material may be used for the passivation layers, such as titanium oxide and silicon oxide.

Particles 502 may be formed from any suitable material and may be of any suitable size. Generally, particles 502 are submicron particles. In a particular embodiment, particles 502 are formed from gold; however, any other suitable materials may be utilized. The size, shape, and type of material used for particles 502 may be selected in order to achieve maximum light scattering at minimum optical power loss. Particles 502 may have a spherical or rough shape. According to one embodiment of the invention, Mie theory may be utilized to determine the size and type of material for particles 502.

Modifications, additions, or omissions may be made to semiconductor laser system 112 without departing from the scope of the invention. The components of semiconductor laser system 112 may be integrated or separated according to particular needs. For example, inflatable balloon 114 may not be necessary for many applications. Moreover, the operations of system 112 may be performed by more, fewer, or other modules. Additionally, operations of semiconductor laser system 112 may be performed using any suitable logic comprising software, hardware, other logic, or any suitable combination of the preceding.

Certain embodiments of the invention may include none, some, or all of the above technical advantages. One or more other technical advantages may be readily apparent to one skilled in the art from the figures, descriptions, and claims included herein.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure, as defined by the following claims.

1. A semiconductor device system, comprising:
a chamber;
one or more semiconductor devices disposed within the chamber, the one or more semiconductor devices operable to emit light; and
an insulator fluid disposed within the chamber, the insulator fluid in contact with the one or more semiconductor devices, the insulator fluid operable to decrease the temperature of the one or more semiconductor devices.

2. The semiconductor device system of claim 1, wherein:
the insulator fluid comprises deionized water.

3. The semiconductor device system of claim 1, wherein the chamber comprises:
a flexible conduit, the insulator fluid operable to circulate through the flexible conduit.

4. The semiconductor device system of claim 1, wherein the chamber comprises:
a first flexible conduit; and
a second flexible conduit disposed within the first flexible conduit, the insulator fluid operable to enter the chamber through a flexible conduit of the first and second flexible conduits and to leave the chamber through the other flexible conduit of the first and second flexible conduits.

5. The semiconductor device system of claim 1, further comprising:
one or more focusing elements, a focusing element comprising a surface operable to:
change shape in response to a pressure change in the insulator fluid; and
focus the light emitted from a semiconductor device of the one or more semiconductor devices.

6. The semiconductor device system of claim 1, further comprising:
a cover insertable into a passage, the chamber disposed within the cover.

7. The semiconductor device system of claim 1, further comprising:
a heating element disposed within the chamber, the heating element operable to increase the temperature of an area disposed outwardly from the chamber.

8. The semiconductor device system of claim 1, further comprising:
an oxygen detector disposed within the chamber, the oxygen detector operable to detect oxygen in an area disposed outwardly from the chamber.

9. The semiconductor device system of claim 1, further comprising:
a substrate disposed within the chamber; and
one or more submounts coupled to a surface of the substrate, a semiconductor device of the one or more semiconductor devices coupled to a submount of the one or more of submounts.

10. The semiconductor device system of claim 1, wherein a semiconductor device of the one or more semiconductor devices comprises:
a light scattering element, the light scattering element operable to scatter light emitted from the semiconductor device.

11. A method of cooling a semiconductor device system having a chamber, comprising:
emitting light from one or more semiconductor devices disposed within a chamber; and
circulating an insulator fluid through the chamber, the insulator fluid in direct contact with the one or more semiconductor devices, the insulator fluid operable to decrease the temperature of the one or more semiconductor devices.

12. The method of claim 11, wherein:
the insulator fluid comprises deionized water.

13. The method of claim 11, wherein circulating an insulator fluid further comprises:
circulating the insulator fluid through a flexible conduit disposed within the chamber.

14. The method of claim 11, wherein circulating an insulator fluid further comprises:
directing the insulator fluid to enter the chamber through a flexible conduit of a first and a second flexible conduits, the first flexible conduit disposed within the chamber, the second flexible conduit disposed within the first flexible conduit; and
directing the insulator fluid to leave the chamber through the other flexible conduit of the first and second flexible conduits.

15. The method of claim 11, further comprising:
adjusting pressure of the insulator fluid to change shape of a surface of a focusing element, the surface operable to focus the light emitted from a semiconductor device of the one or more semiconductor devices.

16. The method of claim 11, wherein the chamber is disposed within a cover insertable into a passage.

17. The method of claim 11, further comprising:
increasing the temperature of an area disposed outwardly from the chamber with a heating element disposed within the chamber.

18. The method of claim 11, further comprising:
detecting oxygen in an area disposed outwardly from the chamber with an oxygen detector disposed within the chamber.

19. The method of claim 11, further comprising:
detecting oxygen in an area disposed outwardly from the chamber with an oxygen detector; and
in response to detecting oxygen, adjusting the light emitted from one of the one or more semiconductor devices.

20. The method of claim 11, wherein a semiconductor device of the one or more semiconductor devices is coupled to a submount, the submount coupled to a surface of a substrate disposed within the chamber.

21. The method of claim 11, further comprising:
scattering the light emitted from one of the one or more semiconductor devices with a light scattering element.

22. A system for cooling a semiconductor device system having a chamber, comprising:
a means for emitting light from one or more semiconductor devices disposed within a chamber; and
a means for circulating an insulator fluid through the chamber, the insulator fluid in direct contact with the one or more semiconductor devices, the insulator fluid operable to decrease the temperature of the one or more semiconductor devices.

23. A semiconductor device system, comprising:
a chamber comprising:
a first flexible conduit; and
a second flexible conduit disposed within the first flexible conduit;
one or more semiconductor devices disposed within the chamber, the one or more semiconductor devices operable to emit light;
an insulator fluid in contact with the one or more semiconductor devices, the insulator fluid operable to decrease the temperature of the one or more semiconductor devices, the insulator fluid comprising deionized water, the insulator fluid operable to enter the chamber through a flexible conduit of the first and second flexible conduits and to leave the chamber through the other flexible conduit of the first and second flexible conduits;
one or more focusing elements, a focusing element comprising a surface operable to:
change shape in response to a pressure change in the insulator fluid; and
focus the light emitted from a semiconductor device of the one or more semiconductor devices;
a cover insertable into a passage, the chamber disposed within the cover;
a heating element disposed within the chamber, the heating element operable to increase the temperature of an area disposed outwardly from the chamber;
an oxygen detector disposed within the chamber, the oxygen detector operable to detect oxygen in an area disposed outwardly from the chamber;
a substrate disposed within the chamber;
one or more submounts coupled to a surface of the substrate, a semiconductor device of the one or more semiconductor devices coupled to a submount of the one or more of submounts; and
a light scattering element, the light scattering element operable to scatter light emitted from the semiconductor device.
